(19) United States
(12) Patent Application Publication
(10) Pub. No.: US 2024/0325270 A1
(43) Pub. Date: Oct. 3, 2024

(54) PHOTOCURABLE COMPOSITION FOR NAIL OR ARTIFICIAL NAIL

(71) Applicant: THREEBOND CO., LTD., Hachioji-shi, Tokyo (JP)

(72) Inventor: Natsumi HARADA, Hachioji-shi, Tokyo (JP)

(21) Appl. No.: 18/708,535

(22) PCT Filed: Oct. 31, 2022
(86) PCT No.: PCT/JP2022/040731

§ 371 Date: May 8, 2024

(30) Foreign Application Priority Data
Nov. 12, 2021 (JP) 2021-184529

(51) Int. Cl. A61K 8/58; A61K 8/46; A61K 8/81; A61Q 3/02; C08K 3/36; C08K 5/37; C08K 9/06

(52) U.S. Cl. CPC A61K 8/585; A61K 8/46; A61K 8/8152; A61Q 3/02; C08K 3/36; C08K 5/37; C08K 9/06

(57) ABSTRACT

A photocurable composition for a nail or an artificial nail, contains a polythiol compound and fumed silica, in which the composition allows both a high structural viscosity ratio (high thixotropy), and a high transparency (low turbidity) of a cured material to be achieved. A photocurable composition for a nail or an artificial nail, contains Component (A): a compound having a (meth)acryloyl group; Component (B): a polythiol compound; Component (C): a photoinitiator; and Component (D): a filling agent containing a component (D-1) and a component (D-2). The component (D-1) and the component (D-2) are each surface-treated fumed silica having a specific residue on the surface thereof.

### TECHNICAL FIELD

The present invention relates to a photocurable composition for a nail or an artificial nail, containing a polythiol compound.

### BACKGROUND ART

It is known in the field of a gel nail for photocuring that fumed silica is added to a photocurable composition for a nail or an artificial nail in order to enhance thixotropy (for example, Japanese Patent Laid-Open No. 2010-013439).

### SUMMARY OF INVENTION

However, in a case where fumed silica is added to a photocurable composition for a nail or an artificial nail, no thixotropy may be exhibited depending on the type of a raw material contained in the composition or depending on a combination of the raw material and fumed silica. Furthermore, a problem is that an increase in amount of addition of fumed silica for an enhancement in thixotropy leads to a reduction in transparency of a cured material.

Thus, a photocurable composition for a nail or an artificial nail, containing a polythiol compound, has been conventionally difficult to allow both a high structural viscosity ratio (high thixotropy), and a high transparency (low turbidity) of a cured material to be achieved, if containing fumed silica added thereto.

An object of the present invention is to provide a photocurable composition for a nail or an artificial nail, containing a polythiol compound and fumed silica, in which the composition allows both a high structural viscosity ratio (high thixotropy), and a high transparency (low t-Urbidity) of a cured material to be achieved.

The present inventors have made intensive studies in order to achieve the above object, and as a result, have completed the present invention relating to a photocurable composition for a nail or an artificial nail.

The gist of the present invention is then described. A first aspect of the present invention relates to a photocurable composition for a nail or an artificial nail, containing the following components (A) to (D):
Component (A): a compound having a (meth) acryloyl group
Component (B): a polythiol compound
Component (C): a photoinitiator
Component (D): a filling agent containing a component (D-1) and a component (D-2)
Component (D-1): surface-treated fumed silica where a residue on the surface is represented by the following formula 1:

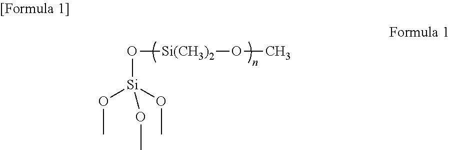

wherein n is an integer of 1 or more; Component (D-2): surface-treated fumed silica where a residue on the surface is represented by the following formula 2.

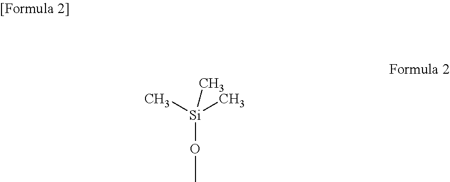

A second aspect of the present invention relates to the photocurable composition for a nail or an artificial nail according to the first aspect, wherein a mass ratio of the component (D-1) and the component (D-2) (component (D-1): component (D-2)) in the entire component (D) is 20:80 to 80:20.

A third aspect of the present invention relates to the photocurable composition for a nail or an artificial nail according to first aspect or the second aspect, the composition containing 1.0 to 20.0% by mass of the component (D) relative to the entire composition.

A fourth aspect of the present invention relates to the photocurable composition for a nail or an artificial nail according to any of the first aspect to the third aspect, the composition containing 0.1 to 50 parts by mass of the component (B) and 0.1 to 10 parts by mass of the component (C) based on 100 parts by mass of the component (A).

A fifth aspect of the present invention relates to the photocurable composition for a nail or an artificial nail according to any of the first aspect to the fourth aspect, wherein the component (A) contains a (meth)acrylate oligomer and a (meth)acrylate monomer.

A sixth aspect of the present invention relates to the photocurable composition for a nail or an artificial nail according to fifth aspect, wherein the (meth)acrylate monomer consists only of a monofunctional (meth)acrylate monomer and/or a difunctional (meth)acrylate monomer.

A seventh aspect of the present invention relates to the photocurable composition for a nail or an artificial nail according to sixth aspect, wherein the monofunctional (meth)acrylate monomer is a monofunctional (meth)acrylate monomer having a hydroxyl group.

An eighth aspect of the present invention relates to the photocurable composition for a nail or an artificial nail according to sixth aspect or the seventh aspect, wherein the difunctional (meth)acrylate monomer is dimethylol tricyclodecane di(meth)acrylate.

A ninth aspect of the present invention relates to the photocurable composition for a nail or an artificial nail according to any of the first aspect to the eighth aspect, for use in an art gel nail.

### DESCRIPTION OF EMBODIMENTS

Hereinafter, embodiments of the present invention are described. The present invention is not limited only to the following embodiments. Herein, operations, and measurement of physical properties and the like are carried out in conditions of room temperature (20° C. or more and 25° C. or less) and a relative humidity of 40% RH or more and 50% RH or less, unless particularly noted. Herein, the photocurable composition for a nail or an artificial nail is also simply referred to as “photocurable composition” or “composition”.

One embodiment of the present invention relates to a photocurable composition for a nail or an artificial nail, containing the following components (A) to (D):
Component (A): a compound having a (meth) acryloyl group
Component (B): a polythiol compound
Component (C): a photoinitiator
Component (D): a filling agent containing a component (D-1) and a component (D-2)
Component (D-1): surface-treated fumed silica where a residue on the surface is represented by the following formula 1:

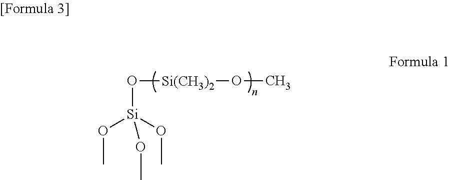

wherein n is an integer of 1 or more;

Component (D-2): surface-treated fumed silica where a residue on the surface is represented by the following formula 2.

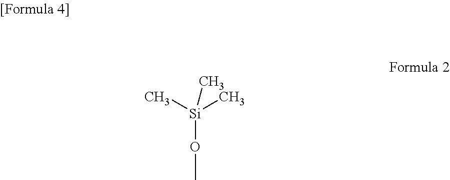

According to the present invention, it is possible for a photocurable composition for a nail or an artificial nail, containing a polythiol compound and fumed silica, to allow both a high structural viscosity ratio, and a high transparency (low turbidity) of a cured material to be achieved. Accordingly, the photocurable composition of the present invention is particularly suitable for an art gel nail for formation of a cubic decoration.

The detail of the present invention is then described. The component (A) usable in the present invention can be any compound having a (meth)acryloyl group. The component (A) here used can be specifically a compound such as (meth)acrylate or (meth)acrylamide. The component (A) also encompasses a (meth)acrylate monomer and a (meth)acrylate oligomer. In the present invention, “acrylic” and “methacrylic” are collectively called “(meth)acrylic”. Herein, the term “(meth)acrylic” encompasses both acrylic and methacrylic. Therefore, for example, the term “(meth)acrylic acid” encompasses both acrylic acid and methacrylic acid. Similarly, the term “(meth)acryloyl” encompasses both acryloyl and methacryloyl. Therefore, for example, the term “(meth)acryloyl group” encompasses both “acryloyl group” and “methacryloyl group”. The component (A) is preferably in the form of a liquid under an atmosphere at 25° C., and can be suitably used as long as it has favorable compatibility with the following component (B) and component (C) in the present invention.

The component (A) preferably contains a (meth)acrylate monomer or a (meth)acrylate oligomer, more preferably contains both a (meth)acrylate monomer and a (meth)acrylate oligomer. In a preferred embodiment of the present invention, the component (A) consists of a (meth)acrylate monomer and a (meth)acrylate oligomer.

Specific examples of the (meth)acrylate oligomer include a (meth)acrylate oligomer having an ester bond in its molecule, a (meth)acrylate oligomer having an ether bond in its molecule, a (meth)acrylate oligomer having a urethane bond in its molecule (urethane (meth)acrylate oligomer), and an epoxy-modified (meth)acrylate oligomer, and examples of the main backbone thereof include bisphenol A, novolac phenol, polybutadiene, polyester, and polyether, but not limited thereto. Two or more such (meth)acrylate oligomers may also be used in combination. The component (A) usable in the present invention also encompasses a compound having one or more epoxy groups and one or more (meth)acryloyl groups in one molecule.

As a method for synthesizing the (meth)acrylate oligomer having an ester bond, a synthesis method including reacting a polyol and a polyvalent carboxylic acid to form an ester bond and add (meth)acrylic acid to an unreacted hydroxyl group has been known, but not limited to this synthesis method. Specific examples thereof include Aronix M-6100, M-6200, M-6250, M-6500, M-7100, M-7300K, M-8030, M-8060, M-8100, M-8530, M-8560, and M-9050 manufactured by Toagosei Co., Ltd., and UV-3500BA, UV-3520TL, UV-3200B, and UV-3000B manufactured by Nippon Synthetic Chemical Industry Co., Ltd., but not limited thereto.

As a method for synthesizing the (meth)acrylate oligomer having an ether bond is a synthesis method including adding (meth)acrylic acid to a hydroxyl group of a polyether polyol, or a hydroxyl group of a polyether polyol having an aromatic such as bisphenol has been known, but not limited to this synthesis method. Specific examples thereof include UV-6640B, UV-6100B, and UV-3700B manufactured by Nippon Synthetic Chemical Industry Co., Ltd., Light (meth)acrylates 3EG-A, 4EG-A, 9EG-A, 14EG-A, PTMGA-250, BP-4EA, BP-4PA, and BP-10EA manufactured by Kyoeisha Chemical Co., Ltd., and EBECRYL3700 manufactured by Daicel-Cytec Co., Ltd., but not limited thereto.

As a method for synthesizing the (meth)acrylate oligomer having a urethane bond is, for example, a synthesis method including forming a urethane bond by a polyol and a polyisocyanate and adding a compound having a hydroxyl group and a (meth)acryloyl group in its molecule, or (meth)acrylic acid to the remaining isocyanate group has been known. The (meth)acrylate oligomer having a urethane bond is preferably added from the viewpoint of an enhancement in durability. Specific examples thereof include AH-600, AT-600, UA-306H, and UF-8001G manufactured by Kyoeisha Chemical Co., Ltd., KY-11 manufactured by Negami Chemical Industrial Co., Ltd., and RUA-075 manufactured by Asia Industry Co., Ltd., but not limited thereto.

The epoxy-modified (meth)acrylate oligomer can be synthesized by subjecting a glycidyl group of a polyfunctional glycidyl ether compound to ring-opening polymerization with (meth)acrylic acid or the like, without limitation to such synthesis. Various types of backbones such as a bisphenol A type, a bisphenol F type, and a novolac phenol type can be used in a main chain of such a polyfunctional glycidyl ether. Specific examples of the epoxy-modified (meth)acrylate oligomer include epoxy esters 3000A and 3002A manufactured by Kyoeisha Chemical Co., Ltd., and EBECRYL3700 manufactured by Daicel-Allnex Ltd., but not limited thereto.

A weight average molecular weight of the (meth)acrylate oligomer is not particularly limited, and is preferably 1000 to 50000, more preferably 1000 to 10000. A weight average molecular weight of 1000 or more can allow a cured material to exhibit toughness, and a weight average molecular weight of 50000 or less can allow the viscosity of the composition to be kept low. The weight average molecular weight here refers to a weight average molecular weight in terms of polystyrene, measured by gel permeation chromatography. In a case where two or more such (meth)acrylate oligomers are used, preferably at least one thereof has a weight average molecular weight in the above range, and more preferably all thereof have a weight average molecular weight in the above range. The (meth)acrylate oligomer preferably contains three or more (meth)acryloyl groups (tri- or higher functional) in one molecule. The (meth)acrylate oligomer is not particularly limited, and preferably contains five or less (meth)acryloyl groups (penta- or lower functional) in one molecule.

The (meth)acrylate monomer can include monofunctional, difunctional, or trifunctional (meth)acrylate monomer and (meth)acrylamide monomer. Two or more such (meth)acrylate monomers may also be used in combination. A plurality of other (meth)acrylate monomers can also be used in combination. The molecular weight of such a (meth)acrylate monomer is not particularly limited, and is, for example, less than 1000.

The component (A) in the photocurable composition of the present embodiment preferably contains a monofunctional (meth)acrylate monomer or a difunctional (meth)acrylate monomer as the (meth)acrylate monomer, more preferably contains both a monofunctional (meth)acrylate monomer and a difunctional (meth)acrylate monomer.

In a preferred embodiment of the present invention, the (meth)acrylate monomer consists only of a monofunctional (meth)acrylate monomer and/or a difunctional (meth)acrylate monomer. In a preferred embodiment of the present invention, the (meth)acrylate monomer consists only of a monofunctional (meth)acrylate monomer and a difunctional (meth)acrylate monomer. Thus, the effects of the present invention can be further remarkably obtained.

Specific examples of the monofunctional (meth)acrylate monomer include lauryl (meth)acrylate, stearyl (meth)acrylate, ethyl carbitol (meth)acrylate, tetrahydrofurfuryl (meth)acrylate, caprolactone-modified tetrahydrofurfuryl (meth)acrylate, cyclohexyl (meth)acrylate, dicyclopentanyl (meth)acrylate, isobornyl (meth)acrylate, benzyl (meth)acrylate, phenyl (meth)acrylate, phenoxyethyl (meth)acrylate, phenoxydiethylene glycol (meth)acrylate, phenoxytetraethylene glycol (meth)acrylate, nonylphenoxyethyl (meth)acrylate, nonylphenoxytetraethylene glycol (meth)acrylate, methoxydiethylene glycol (meth)acrylate, ethoxydiethylene glycol (meth)acrylate, butoxyethyl (meth)acrylate, butoxytriethylene glycol (meth)acrylate, 2-ethylhexylpolyethylene glycol (meth)acrylate, 4-hydroxybutyl (meth)acrylate, nonylphenylpolypropylene glycol (meth)acrylate, methoxydipropylene glycol (meth)acrylate, 2-hydroxyethyl (meth)acrylate, 2-hydroxypropyl (meth)acrylate, glycerol (meth)acrylate, polyethylene glycol (meth)acrylate, polypropylene glycol (meth)acrylate, epichlorohydrin-modified butyl (meth)acrylate, epichlorohydrin-modified phenoxy (meth)acrylate, N,N-dimethylaminoethyl (meth)acrylate, and N,N-diethylaminoethyl (meth)acrylate, but not limited thereto. The component (A) preferably contains a monofunctional (meth)acrylate monomer having a hydroxyl group. Thus, the effects of the present invention can be further remarkably obtained. Specific examples of the monofunctional (meth)acrylate monomer having a hydroxyl group include 2-hydroxyethyl (meth)acrylate, 2-hydroxypropyl (meth)acrylate, and 4-hydroxybutyl (meth)acrylate, but not limited thereto.

Examples of the monofunctional (meth)acrylate monomer also include a (meth)acrylate monomer having an acidic group, in addition to the above. Examples of the (meth)acrylate monomer having an acidic group particularly include carboxylic acid or phosphoric acid having a (meth)acryloyl group in its molecule. Examples of the carboxylic acid having a (meth)acryloyl group in its molecule include (meth)acrylic acid, 3-(meth)acryloyloxypropylsuccinic acid, 4-(meth)acryloyloxybutylsuccinic acid, 2-(meth)acryloyloxyethylmaleic acid, 3-(meth)acryloyloxypropylmaleic acid, 4-(meth)acryloyloxybutylmaleic acid, 2-(meth)acryloyloxyethylhexahydrophthalic acid, 3-(meth)acryloyloxypropylhexahydrophthalic acid, 4-(meth)acryloyloxybutylhexahydrophthalic acid, 2-(meth)acryloyloxyethylphthalic acid, 3-(meth)acryloyloxypropylphthalic acid, and 4-(meth)acryloyloxybutylphthalic acid. Examples of the phosphoric acid having a (meth)acryloyl group in its molecule include 2-ethylhexyl acid phosphate, 2-hydroxyethyl methacrylate acid phosphate, and dibutyl phosphate, but not limited thereto. The component (A) preferably contains the (meth)acrylate monomer having an acidic group from the viewpoint of an enhancement in durability.

Specific examples of the difunctional (meth)acrylate monomer include 1,3-butylene glycol di(meth)acrylate, 1,4-butylene glycol di(meth)acrylate, dimethylol tricyclodecane di(meth)acrylate, neopentyl glycol di(meth)acrylate, 1,6-hexane glycol di(meth)acrylate, ethylene glycol di(meth)acrylate, tetraethylene glycol di(meth)acrylate, polyethylene glycol di(meth)acrylate, propylene glycol di(meth)acrylate, tripropylene glycol di(meth)acrylate, ethylene oxide-modified neopentyl glycol di(meth)acrylate, propylene oxide-modified neopentyl glycol di(meth)acrylate, bisphenol A di(meth)acrylate, ethylene oxide-modified bisphenol A di(meth)acrylate, epichlorohydrin-modified bisphenol A di(meth)acrylate, ethylene oxide-modified bisphenol S di(meth)acrylate, neopentyl glycol-modified trimethylolpropane di(meth)acrylate, dicyclopentenyl di(meth)acrylate, ethylene oxide-modified dicyclopentenyl di(meth)acrylate, and di(meth)acryloyl isocyanurate, but not limited thereto. In particular, the difunctional (meth)acrylate monomer is preferably dimethylol tricyclodecane di(meth)acrylate, more preferably dimethylol tricyclodecane diacrylate. Thus, the effects of the present invention can be further remarkably obtained.

Specific examples of the trifunctional (meth)acrylate monomer include trimethylolpropane tri(meth)acrylate, trimethylolpropane tri(meth)acrylate, pentaerythritol tri(meth)acrylate, ethylene oxide (EO)-modified trimethylolpropane tri(meth)acrylate, propylene oxide (PO)-modified trimethylolpropane tri(meth)acrylate, epichlorohydrin (ECH)-modified trimethylolpropane tri(meth)acrylate, ECH-modified glycerol tri(meth)acrylate, tris(acryloyloxyethyl)isocyanurate, and caprolactone-modified tris(2-acryloyloxyethyl)isocyanurate, but not limited thereto.

Specific examples of the (meth)acrylamide monomer include dimethyl(meth)acrylamide, (meth)acryloylmorpholine, and diethyl(meth)acrylamide, but not limited thereto. Although no clear reason is found, the monomer preferably contains the (meth)acrylamide monomer from the viewpoint of an enhancement in durability. DMAA, ACMO, DEAA, and the like manufactured by KJ Chemicals Corporation are known as specific examples of the (meth)acrylamide monomer in the present invention, without limitation thereto.

Specific examples of other (meth)acrylate monomer include a tetra- or higher functional (meth)acrylate monomer such as dipentaerythritol hexa(meth)acrylate.

The component (A) preferably contains both the (meth)acrylate oligomer and the (meth)acrylate monomer. Here, the ratio of the contents of the (meth)acrylate oligomer and the (meth)acrylate monomer (mass ratio of (meth)acrylate oligomer:(meth)acrylate monomer) is preferably 50:50 to 95:5, more preferably 60:40 to 95:5, further preferably 70:30 to 95:5, preferably 70:30 to 80:20. In a case where the photocurable composition contains two or more such (meth)acrylate oligomers, the total amount of such oligomers is intended to be the above content. Similarly, in a case where the photocurable composition contains two or more such (meth)acrylate monomers, the total amount of such monomers is intended to be the above content. The photocurable composition contains the (meth)acrylate oligomer and thus exhibit enhanced durability.

The (meth)acrylate monomer is preferably a combination of the monofunctional (meth)acrylate monomer and a di- or higher functional (meth)acrylate monomer. In the case of such combination use of the monofunctional (meth)acrylate monomer and the di- or higher functional (meth)acrylate monomer, the ratio of the contents thereof (mass ratio of monofunctional (meth)acrylate monomer:di— or higher functional (meth)acrylate monomer) is not particularly limited, and is, for example, 95:5 to 5:95, preferably 80:20 to 50:50, more preferably 70:30 to 60:40. In a case where the photocurable composition contains two or more such monofunctional (meth)acrylate monomers, the total amount of such monomers is intended to be the above content. Similarly, in a case where the photocurable composition contains two or more di— or higher functional (meth)acrylate monomers, the total amount of such monomers is intended to be the above content.

The component (B) usable in the present invention is a polythiol compound. The component (B) is not particularly limited as long as it is a compound having two or more thiol groups. The polythiol compound may be used singly or in combinations of two or more kinds thereof. The component (B) is added to cause no oxygen inhibition and improve surface curability. Specific examples of the component (B) include an aliphatic polythiol compound and an aromatic polythiol compound, but not limited thereto. The aliphatic polythiol compound and the aromatic polythiol compound may be each a polythiol compound having a sulfide bond or a polythiol compound having a secondary thiol group.

Examples of an aliphatic polythiol compound having two thiol groups include 1,2-ethanedithiol, 1,2-propanedithiol, 1,3-propanedithiol, 1,4-butanedithiol, 1,6-hexanedithiol, 1,7-heptanedithiol, 1,8-octanedithiol, 1,9-nonanedithiol, 1,10-decanedithiol, 1,12-dodecanedithiol, 2,2-dimethyl-1,3-propanedithiol, 3-methyl-1,5-pentanedithiol, 2-methyl-1,8-octanedithiol, 1,4-cyclohexanedithiol, 1,4-bis(mercaptomethyl)cyclohexane, 1,1-cyclohexanedithiol, 1,2-cyclohexanedithiol, bicyclo[2,2,1]hepta-exo-cis-2,3-dithiol, 1,1-bis(mercaptomethyl)cyclohexane, bis(2-mercaptoethyl)ether, ethylene glycol bis(2-mercaptoacetate), and ethylene glycol bis(3-mercaptopropionate), but not limited thereto.

Examples of an aliphatic polythiol compound having three thiol groups include 1,1,1-tris(mercaptomethyl)ethane, 2-ethyl-2-mercaptomethyl-1,3-propanedithiol, 1,2,3-propanetrithiol, trimethylolpropane tris(2-mercaptoacetate), trimethylolpropane tris(3-mercaptopropionate), and tris[(mercaptopropynyloxy)-ethyl]isocyanurate, but not limited thereto.

Examples of an aliphatic polythiol compound having four or more thiol groups include pentaerythritol tetrakis(2-mercaptoacetate), pentaerythritol tetrakis(3-mercaptopropionate), pentaerythritol tetrakis(3-mercaptobutyrate), and dipentaerythritol hexa-3-mercaptopropionate, but not limited thereto.

Examples of the aromatic polythiol compound include 1,2-dimercaptobenzene, 1,3-dimercaptobenzene, 1,4-dimercaptobenzene, 1,2-bis(mercaptomethyl)benzene, 1,3-bis(mercaptomethyl)benzene, 1,4-bis(mercaptomethyl)benzene, 1,2-bis(2-mercaptoethyl)benzene, 1,3-bis(2-mercaptoethyl)benzene, 1,4-bis(2-mercaptoethyl)benzene, 1,2-bis(2-mercaptoethyleneoxy)benzene, 1,3-bis(2-mercaptoethyleneoxy)benzene, 1,4-bis(2-mercaptoethyleneoxy)benzene, 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(2-mercaptoethyl)benzene, 1,2,4-tris(2-mercaptoethyl)benzene, 1,3,5-tris(2-mercaptoethyl)benzene, 1,2,3-tris(2-mercaptoethyleneoxy)benzene, 1,2,4-tris(2-mercaptoethyleneoxy)benzene, 1,3,5-tris(2-mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(2-mercaptoethyl)benzene, 1,2,3,5-tetrakis(2-mercaptoethyl)benzene, 1,2,4,5-tetrakis(2-mercaptoethyl)benzene, 1,2,3,4-tetrakis(2-mercaptoethyleneoxy)benzene, 1,2,3,5-tetrakis(2-mercaptoethyleneoxy)benzene, 1,2,4,5-tetrakis(2-mercaptoethyleneoxy)benzene, 2,2′-mercaptobiphenyl, 4,4′-thiobis-benzenethiol, 4,4′-dimercaptobiphenyl, 4,4′-dimercaptobibenzyl, 2,5-toluenedithiol, 3,4-toluenedithiol, 1,4-naphthalenedithiol, 1,5-naphthalenedithiol, 2,6-naphthalenedithiol, 2,7-naphthalenedithiol, 2,4-dimethylbenzene-1,3-dithiol, 4,5-dimethylbenzene-1,3-dithiol, 9,10-anthracenedimethanethiol, 1,3-bis(2-mercaptoethylthio)benzene, 1,4-bis(2-mercaptoethylthio)benzene, 1,2-bis(2-mercaptoethylthiomethyl)benzene, 1,3-bis(2-mercaptoethylthiomethyl)benzene, 1,4-bis(2-mercaptoethylthiomethyl)benzene, 1,2,3-tris(2-mercaptoethylthio)benzene, 1,2,4-tris(2-mercaptoethylthio)benzene, 1,3,5-tris(2-mercaptoethylthio)benzene, 1,2,3,4-tetrakis(2-mercaptoethylthio)benzene, 1,2,3,5-tetrakis(2-mercaptoethylthio)benzene, and 1,2,4,5-tetrakis(2-mercaptoethylthio)benzene, but not limited thereto.

Examples of the polythiol compound having a sulfide bond include bis(2-mercaptoethyl)sulfide, bis(2-mercaptoethylthio)methane, 1,2-bis(2-mercaptoethylthio)ethane, 1,3-bis(2-mercaptoethylthio)propane, 1,2,3-tris(2-mercaptoethylthio)propane, tetrakis(2-mercaptoethylthiomethyl)methane, 1,2-bis(2-mercaptoethylthio)propanethiol, 2,5-dimercapto-1,4-dithiane, bis(2-mercaptoethyl)disulfide, 3,4-thiophenedithiol, 1,2-bis(2-mercaptoethyl)thio-3-mercaptopropane, and bis-(2-mercaptoethylthio-3-mercaptopropane)sulfide, but not limited thereto.

Specific examples of the polythiol compound having a secondary thiol group include pentaerythritol tetrakis(3-mercaptobutyrate), 1,4-bis(3-mercaptobutyryloxy)butane, 1,3,5-tris(3-mercaptobutyryloxyethyl)-1,3,5-triazin-2,4,6(1H,3H,5H)-trione, trimethylolpropane tris(3-mercaptobutyrate), trimethylolethane tris(3-mercaptobutyrate), trimethylolpropane tris(3-mercaptobutyrate), and trimethylolethane tris(3-mercaptobutyrate), but not limited thereto. Examples of a commercial product include PEMP manufactured by SC Organic Chemical Co., Ltd., and KarenzMT (registered trademark) series, PE1, BD1, and NR1 manufactured by Showa Denko K.K., but not limited thereto.

The content of the component (B) in the photocurable composition is not particularly limited, and the content of the component (B) is preferably 0.1 to 50 parts by mass, more preferably 1 to 30 parts by mass, further preferably 1 to 10 parts by mass, still more preferably 1 to 5 parts by mass based on 100 parts by mass of the component (A). The content of the component (B) is 0.1 parts by mass or more to result in an enhancement in surface curability, and is 50 parts by mass or less to result in an enhancement in storage stability. In a case where the photocurable composition contains two or more such polythiol compounds in the component (B), the total amount of such compounds is intended to be the above content. Similarly, in a case where the photocurable composition contains two or more such compounds having a (meth)acryloyl group in the component (A), the total amount of such compounds is intended to be the above content.

The component (C) usable in the present invention is a photoinitiator. The component (C) is not limited as long as it is a radical photoinitiator which generates radical species by an energy ray such as visible light, ultraviolet light, X-ray, or electron beam.

Specific examples of the component (C), which is a non-visible light type photoinitiator, include acetophenones such as diethoxyacetophenone, 2-hydroxy-2-methyl-1-phenylpropan-1-one, benzyl dimethyl ketal, 4-(2-hydroxyethoxy)phenyl-(2-hydroxy-2-propyl)ketone, 1-hydroxycyclohexyl phenyl ketone, 2-methyl-2-morpholino(4-thiomethylphenyl)propan-1-one, 2-benzyl-2-dimethylamino-1-(4-morpholino phenyl)butanone, and a 2-hydroxy-2-methyl-1-[4-(1-methylvinyl)phenyl]propanone oligomer; benzoins such as benzoin, benzoin methyl ether, benzoin ethyl ether, benzoin isopropyl ether, and benzoin isobutyl ether; benzophenones such as benzophenone, methyl o-benzoylbenzoate, 4-phenylbenzophenone, 4-benzoyl-4′-methyl-diphenyl sulfide, 3,3′,4,4′-tetra(t-butylperoxycarbonyl)benzophenone, 2,4,6-trimethylbenzophenone, 4-benzoyl-N,N-dimethyl-N-[2-(1-oxo-2-propenyloxy)ethyl]benzenemethanaminium bromide, and (4-benzoylbenzyl)trimethylammonium chloride; and thioxanthones such as 2-isopropylthioxanthone, 4-isopropylthioxanthone, 2,4-diethylthioxanthone, 2,4-dichlorothioxanthone, 1-chloro-4-propoxythioxanthone, and 2-(3-dimethylamino-2-hydroxy)-3,4-dimethyl-9H-thioxanthon-9-one-mesochloride, but not limited thereto. The component (C) can also be used in combinations thereof.

The component (C) preferably contains a visible light type photoinitiator. The content of the visible light type photoinitiator (in the case of two or more kinds, the total content) relative to the entire component (C) is, for example, 0 to 70% by mass, preferably more than 0 and 70% by mass or less, more preferably 10 to 60% by mass, further preferably 20 to 50% by mass. The visible light type photoinitiator is contained to hardly cause yellowing of a cured material.

The visible light type photoinitiator is here a photoinitiator which most strongly absorbs light in the visible region, and, in particular, an acyl phosphine oxide-based photopolymerization initiator containing a phosphorus atom can be preferably used. Specific examples of the visible light type photoinitiator include 2,4,6-trimethylbenzoyl-diphenyl-phosphine oxide and bis(2,4,6-trimethylbenzoyl)-phenylphosphine oxide, but not limited thereto.

The content of the component (C) in the photocurable composition is not particularly limited, and the content of the component (C) is preferably 0.1 to 20 parts by mass, more preferably 0.1 to 10 parts by mass, further preferably 1 to 10 parts by mass based on 100 parts by mass of the component (A). When the content of the component (C) is 0.1 parts by mass or more, photocurability can be maintained. On the other hand, when the content of the component (C) is 20 parts by mass or less, storage stability can be maintained without thickening during storage. In a case where the photocurable composition contains two or more photoinitiators in the component (C), the total amount of such photoinitiators is intended to be the above content. Similarly, in a case where the photocurable composition contains two or more compounds each having a (meth)acryloyl group, in the component (A), the total amount of such compounds is intended to be the above content.

The component (D) usable in the present invention is a filling agent containing a component (D-1) and a component (D-2). The component (D-1) is surface-treated fumed silica where a residue on the surface is represented by the following formula 1, and the component (D-2) is surface-treated fumed silica where a residue on the surface is represented by the following formula 2. Such fumed silica is obtained by a dry method, and is specifically silicon dioxide obtained by hydrolysis in a flame of oxygen and hydrogen with silicon tetrachloride as a raw material. Herein, an aggregate of such silica can be formed in the photocurable composition. While fumed silica after production is in a state where silanol (═SiOH) is exposed, surface-treated fumed silica can be obtained due to surface treatment by reaction of a compound reactive with silanol on the surface of the fumed silica (chemical modification). The component (D) is added to enable the viscosity and thixotropy to be controlled.

The component (D) preferably consists only of the component (D-1) and the component (D-2), and the composition most preferably contains no filling agent other than the component (D-1) and the component (D-2). For example, the total content of the component (D-1) and the component (D-2) in the entire filling agent contained in the photocurable composition is preferably 95% by mass or more, more preferably 99% by mass or more, most preferably 100% by mass.

The surface-treated fumed silica of the component (D-1) is fumed silica where a residue on the surface has a structure represented by the following formula 1, by chemical modification of the surface of the fumed silica. Here, n is an integer of 1 or more. n is, for example, an integer of 2 or more. The upper limit value of n is not particularly limited, and is, for example, 1000 or less, for example, 100 or less. The component (D-1) can be, for example, fumed silica surface-treated with polydimethylsiloxane.

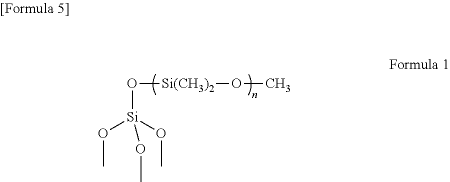

The surface-treated fumed silica of the component (D-2) is fumed silica where a residue on the surface has a structure represented by the following formula 2, by chemical modification of the surface of the fumed silica. The component (D-2) can be, for example, fumed silica surface-treated with hexamethyldisilazane.

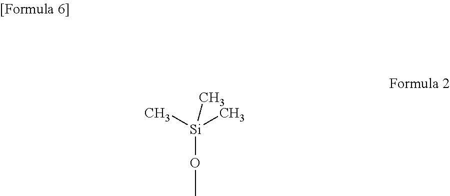

The mass ratio of the component (D-1) and the component (D-2) (component (D-1): component (D-2)) in the entire component (D) is not particularly limited, and is preferably 20:80 to 80:20, more preferably 30:70 to 70:30, further preferably 33:67 to 66:34. Thus, thixotropy, and transparency of a cured material can be achieved at higher levels. In a case where two or more such components (D-1) are contained, the total content thereof is intended to be the content of the component (D-1). Similarly, in a case where two or more such components (D-2) are contained, the total content thereof is intended to be the content of the component (D-2). In particular, in a case where the component (D) consists only of the component (D-1) and the component (D-2), the mass ratio of the component (D-1) and the component (D-2) in the entire component (D) is preferably in the above range. Thus, the effects of the present invention can be more remarkably obtained.

The BET specific surface area of the component (D) is not particularly limited, and is preferably 10 to 500 m²/g, more preferably 10 to 300 m²/g, further preferably 100 to 250 m²/g. The particle diameter of the component (D) is not particularly limited, and the average primary particle diameter of the component (D) is, for example, 1 to 1000 nm. Here, the average primary particle diameter is determined as the average value of the particle diameters confirmed of primary particles randomly extracted with an electron microscope. The average primary particle diameter of the component (D) is preferably 1 to 50 nm. In a case where the composition contains fumed silica having an average primary particle diameter of 1 to 50 nm, the fumed silica having an average primary particle diameter of 1 to 50 nm preferably contains no fumed silica other than the component (D) (component (D-1) and component (D-2)). The photocurable composition of the present embodiment preferably contains no fumed silica other than the component (D) (component (D-1) and component (D-2)).

Specific examples of the component (D-1) include Aerosil (registered trademark) series, RY50, RY51, NY50, NY50L, RY200S, R202, RY200, RY200L, and RY300 manufactured by Nippon Aerosil Co., Ltd., and TS-720 manufactured by CABOT Japan K.K., but not limited thereto. The component (D-1) may be used singly or as a mixture of two or more kinds.

Specific examples of the component (D-2) include Aerosil (registered trademark) series, RX50, NAX50, NX90G, NX90S, NX130, RX200, R8200, RX300, R812, and R812S manufactured by Nippon Aerosil Co., Ltd., but not limited thereto. The component (D-2) may be used singly or as a mixture of two or more kinds.

The content of the component (D) in the photocurable composition is not particularly limited, and the content of the component (D) is preferably 1.0 to 25 parts by mass, more preferably 1.0 to 10 parts by mass based on 100 parts by mass of the component (A). A content of 1.0 part by mass or more can result in a higher structural viscosity ratio, and a content of 25 parts by mass or less can result in a reduction in haze of a cured material (make a cured material transparent). The content of the component (D) relative to the entire composition is preferably 1.0 to 20.0% by mass, more preferably 1.0 to 10% by mass. In a case where the component (D) consists only of the component (D-1) and the component (D-2), the contents of the component (D-1) and the component (D-2) are preferably respectively 1.0 to 6.0 parts by mass and 0.1 to 6.0 parts by mass based on 100 parts by mass of the component (A). In a case where a plurality of such components (D) is contained, the total amount thereof is intended to be the above content. Similarly, in a case where a plurality of such components (A) is contained, the total amount thereof is intended to be the above content.

A proper amount of additive(s) such as a coupling agent, an inorganic filling agent other than the component (D), an organic filling agent, a colorant such as a pigment or a dye, an antioxidant, a polymerization inhibitor, a defoaming agent, a leveling agent, and/or a rheology control agent may be compounded in the photocurable composition of the present invention, as long as features of the present invention are not impaired. Such an additive is added to obtain a composition or a cured material thereof excellent in photocurability, resin strength, adhesion strength, workability, storage stability, and the like.

A coupling agent can be compounded in the photocurable composition of the present invention, as long as features of the present invention are not impaired. Examples of the coupling agent include a silane-based coupling agent having an epoxy group, a vinyl group, an acryloyl group or a methacryloyl group, and also a hydrolyzable silane group in combination, a polyorganosiloxane having a phenyl group and a hydrolyzable silyl group, and/or a polyorganosiloxane having an epoxy group and a hydrolyzable silyl group, but not limited thereto. Specific examples of the silane-based coupling agent include allyltrimethoxysilane, vinyltrimethoxysilane, 3-glycidoxypropyltrimethoxysilane, 3-methacryloxypropyltrimethoxysilane, 3-acryloxypropyltrimethoxysilane, and 3-chloropropyltrimethoxysilane, but not limited thereto.

A filling agent such as an inorganic filling agent other than the component (D), an organic filling agent can be compounded in the photocurable composition of the present invention, as long as features of the present invention are not impaired. Such a filling agent can be compounded to adjust not only viscous properties/thixotropy, but also curability and toughness. Examples of the inorganic filling agent include alumina, silica, fumed silica other than the component (D), a metal powder, and a lame powder, but not limited thereto. On the other hand, examples of the organic filling agent include a styrene filler, a rubber filler, and a core-shell acrylic filler, but not limited thereto. Specific products of silica include FUSELEX E-1 manufactured by Tatsumori Ltd. and AO-802 manufactured by Admafine, but not limited thereto.

The method for preparing the photocurable composition of the present invention is not particularly limited, and a conventionally known method can be appropriately adopted. For example, the component (A), the component (B), the component (D), and a component optionally added are respectively weighed in predetermined amounts, added to a stirring pot sequentially in any order or simultaneously, and then mixed by use of a mixing means such as a planetary mixer preferably with defoaming in vacuum. Here, production conditions are not particularly limited, and production is preferably performed under light shielding conditions. The mixing temperature is preferably a temperature of 10 to 50° C., and the mixing time is preferably 0.1 to 5 hours. Thereafter, the component (C) is weighed and further added to the stirring pot, and mixed by using a mixing means such as a planetary mixer preferably with defoaming in vacuum, and thus the photocurable resin composition can be obtained. Herein, production conditions are not particularly limited, and production is preferably performed under light shielding conditions.

The viscosity at 25° C. of the photocurable composition of the present invention is not particularly limited and is preferably 100 Pa-s or less, more preferably 80 Pa-s or less. When the viscosity is in the above range, handleability in procedures can be enhanced. The viscosity of the photocurable composition, here adopted, is a value measured by a method described in Examples below. The viscosity of the composition can be adjusted by appropriately selecting the types of materials and the contents of the components (A) to (D).

The structural viscosity ratio of the photocurable composition of the present invention is not particularly limited and is preferably 2.5 to 4.5. When the structural viscosity ratio is in the range, coatability can be enhanced. Moreover, coating can be appropriately controlled, and a cubic decoration is easily made. The structural viscosity ratio of the photocurable composition, here adopted, is a value measured by a method described in Examples below. The structural viscosity ratio of the composition can be adjusted by appropriately selecting the types of materials and the contents of the components (A) to (D).

A cured material can be obtained by curing the photocurable composition of the present invention with an energy ray such as visible light, ultraviolet light, X-ray, or electron beam. The method for producing the cured material is not particularly limited.

For example, a nail or an artificial nail can be subjected to nail procedures by coating such nail or artificial nail with the photocurable composition of the present invention and curing the resultant. In the case of procedures of a human nail, it is preferable before such procedures to perform sanding of the surface of the human nail by a file or the like and then remove any dust, oil content, water content, and the like by a nail dedicated solvent mainly containing ethanol. Preferably, in the case of coating a nail or an artificial nail with the photocurable composition of the present invention, a coating film having a thickness of 100 to 300 μm before curing is formed by a pencil or a brush. A primer may also be used in advance in the coating. The irradiation apparatus used in curing of the photocurable composition is not particularly limited, and a commercially available UV lamp or LED lamp can be used. The wavelength of light for irradiation is, for example, 350 to 400 nm. The irradiation time is 15 seconds to 120 seconds, and is preferably 20 to 70 seconds in consideration of the influence on a finger.

The turbidity of a cured material obtained by curing the photocurable composition (the cured material of the photocurable composition) is not particularly limited and is preferably 5.0 to 16.0%. When the turbidity is in the above range, the cured material is suitable for an art gel nail. The turbidity of the cured material, here adopted, is a value measured by a method described in Examples below. The turbidity of the cured material can be adjusted by appropriately selecting the types of materials and the contents of the components (A) to (D).

A compound having a (meth)acryloyl group is inhibited from being polymerized by the action of oxygen inhibition in a region where the compound is in touch with oxygen. The photocurable composition of the present invention is unlikely to be affected by oxygen inhibition resulting from inclusion of a compound having a (meth)acryloyl group, thus has rapid curability by light irradiation, and thus is suitable for a nail or an artificial nail. While the influence of oxygen inhibition causes an uncured component remaining on the surface to generate stickiness and any procedure (wiping) for wiping off such an uncured component is needed to be made, the photocurable composition of the present invention is unlikely to be affected by oxygen inhibition and can allow for non-wiping requiring no wiping procedure. The photocurable composition of the present invention is favorable in surface curability and thus is particularly suitable for an art gel nail for formation of a cubic decoration.

### EXAMPLES

Next, the present invention is further specifically described with reference to Examples, but the present invention is not limited only to these Examples.

The following components were provided for preparing, for example, a photocurable composition for a nail or an artificial nail (hereinafter, the photocurable composition for a nail or an artificial nail being also simply referred to as “photocurable composition” or “composition”.) Component (A): compound having a (meth)acryloyl group
Polyether-based urethane acrylate oligomer having a weight average molecular weight of 5000 and the number of functional groups of 3 (KY-11 manufactured by Negami Chemical Industrial Co., Ltd.)
Urethane acrylate oligomer having a weight average molecular weight of 4600 and the number of functional groups of 5 (RUA-075 manufactured by Asia Industry Co., Ltd.)
4-hydroxybutylacrylate (4-HBA manufactured by Osaka Organic Chemical Industry Ltd.)
Dimethylol tricyclodecane diacrylate (Light Acrylate DCP-A manufactured by Kyoeisha Chemical Co., Ltd.)
2-Hydroxypropyl methacrylate (HPMA manufactured by Nippon Shokubai Co., Ltd.)
Dipentaerythritol hexaacrylate (DPHA manufactured by Daicel-Allnex Ltd.)
Caprolactone-modified tris-(2-acryloyloxyethyl)isocyanurate (NK Ester A9300-1CL manufactured by Shin-Nakamura Chemical Co., Ltd.)
Component (B): polythiol compound
Trimethylolpropane tris(3-mercaptopropionate) (TMMP-20P manufactured by SC Organic Chemical Co., Ltd.)
Component (C): photoinitiator
1-Hydroxycyclohexyl phenyl ketone (non-visible light type photoinitiator) (Omnirad 184, manufactured by IGM Resins B.V.)
2,4,6-Trimethylbenzoyl-diphenyl-phosphine oxide (visible light type photoinitiator) (Omnirad TPO H, manufactured by IGM Resins B.V.)
Component (D): filling agent consisting of component (D-1) and component (D-2)
Component (D-1): surface-treated fumed silica where a residue on the surface is represented by the formula 1
Fumed silica surface-treated with polydimethylsiloxane (BET specific surface area: 200 m²/g) (TS-720 manufactured by CABOT Japan K.K.)
Component (D-2): surface-treated fumed silica where a residue on the surface is represented by the formula 2
Fumed silica surface-treated with hexamethyldisilazane (BET specific surface area: 140 m²/g) (Aerosil (registered trademark) RX200 manufactured by Nippon Aerosil Co., Ltd.)
Component (D′): fumed silica other than component (D)
Fumed silica not treated (BET specific surface area: 200 m²/g) (Aerosil (registered trademark) 200 manufactured by Nippon Aerosil Co., Ltd.)
Fumed silica surface-treated with octylsilane (BET specific surface area: 150 m²/g) (Aerosil (registered trademark) R805 manufactured by Nippon Aerosil Co., Ltd.)
Fumed silica surface-treated with dimethyldichlorosilane (BET specific surface area: 110 m²/g) (Aerosil (registered trademark) R972 manufactured by Nippon Aerosil Co., Ltd.).

A mixture was prepared by adding 10 parts by mass of the component (D) (or component (D′)) to 100 parts by mass of each raw material described in Table 1, and stirring the resultant by a planetary mixer for 30 minutes under reduced pressure retained, and the following structural viscosity ratio and turbidity of the mixture were measured.

### [Structural Viscosity Ratio of Mixture]

The structural viscosity ratio was measured with a rheometer according to the following specification. The viscosity was measured with HAAKE MARSIII manufactured by Thermo Fisher Scientific Inc. The viscosity at a shear speed of 20 s⁻¹ was defined as viscosity 1, and the viscosity at a shear speed of 2 s⁻¹ was defined as viscosity 2. The numerical value of viscosity 1 was defined as “viscosity (Pa-s)” and the numerical value of viscosity 2/viscosity 1 was defined as “structural viscosity ratio”. In a case where not a viscoelastic body, but dilatancy was obtained, the structural viscosity ratio was not measured and “Dilatancy” was described in Table 1. In a case where the component (D) (or component (D′)) was precipitated regardless of stirring by a planetary mixer, “Separated” was described in the Table.

### [Turbidity of Mixture]

An alkali-free glass plate of 50 mm length×50 mm width×0.7 mm thickness was coated with the composition at a thickness of 0.25 mm, to produce a test piece, measurement with a haze meter NDH2000 manufactured by Nippon Denshoku Industries Co., Ltd. was performed, and the result was defined as “turbidity (%)”. In Table 1, in the case of “Dilatancy” or “Separated”, no measurement was performed and “Not measured” was described.

TABLE 1

| | Raw | Component (D) or component (D′) | | | | |
|---|---|---|---|---|---|---|
| Test item | material | 200 | RX200 | R805 | TS-720 | R972 |
| Structural | KY-11 | 2.5 | 1.9 | 1.7 | 2.1 | 1.1 |
| viscosity | RUA-075 | 2.6 | 3.1 | 2.7 | 4.4 | 2.1 |
| ratio | 4-HBA | Separated | 4.8 | 3.3 | 4.6 | 1.6 |
| | HPMA | Separated | 3.3 | 5.7 | 2.9 | 1.2 |
| | DCP-A | 8.0 | 3.4 | 4.3 | 6.2 | Dilatancy |
| | DPHA | 2.8 | 2.9 | 3.6 | 5.5 | 1.2 |
| | A9300-1CL | 3.6 | 4.2 | 4.3 | 6.4 | 1.0 |
| | TMMP-20P | Dilatancy | 5.7 | 5.9 | 7.3 | 2.0 |
| Turbidity | KY-11 | 0.98 | 2.32 | 2.93 | 16.33 | 1.21 |
| | RUA-075 | 0.23 | 2.26 | 2.21 | 11.22 | 0.80 |
| | 4-HBA | Not measured | 0.16 | 0.25 | 0.38 | 0.15 |
| | HPMA | Not measured | 0.18 | 0.60 | 0.51 | 0.16 |
| | DCP-A | 11.49 | 1.31 | 2.49 | 16.72 | Not measured |
| | DPHA | 2.41 | 0.21 | 1.56 | 11.63 | 0.28 |
| | A9300-1CL | 0.47 | 2.67 | 4.37 | 20.14 | 0.52 |
| | TMMP-20P | Not measured | 5.98 | 8.34 | 42.13 | 3.93 |

With respect to the structural viscosity ratio of the mixture with the component (A) or the component (B) in Table 1, TS-720 as the component (D-1) in the component (D) (or component (D′)) exhibited a high structural viscosity ratio (high thixotropy) TS-720 exhibited a high structural viscosity ratio, with TMMP-20P as a polythiol compound, as compared with a (meth)acrylate oligomer and a (meth)acrylate monomer, and thus it was found that TS-720 exhibited a high structural viscosity ratio in, in particular, a composition containing a polythiol compound. With respect to the turbidity in Table 1, RX200 as the component (D-2) in the component (D) (or component (D′)) exhibited a low t:rbidity (high transparency). A mixture of RX200 with a (meth)acrylate monomer tended to exhibit a low turbidiLy as compared with a mixture thereof with a (meth)acrylate oligomer or a polythiol compound. A mixture of RX200 with 4-HBA, HPMA or DPHA as a (meth)acrylate monomer having no cyclic structure exhibited a particularly low turbidity. The reason for this was considered because, when a (meth)acrylate monomer had a cyclic structure, a methyl group on the surface of a particle of RX200 and the cyclic structure of the (meth)acrylate monomer were sometimes mutually repelled to lead to the haze of the mixture. On the other hand, 200 as the component (D′) was found to allow for no appropriate measurement of the structural viscosity ratio depending on the raw material mixed.

### Examples 1 to 5 and Comparative Examples 1 to 3

### Preparation of photocurable composition

The component (A), the component (B) and the component (D) (or component (D′)) were weighed in a stirring pot and then stirred for 30 minutes with defoaming in vacuum. Finally, the component (C) was weighed and added to the stirring pot and the resultant was stirred for 30 minutes, to obtain a photocurable composition of each of Examples and Comparative Examples. The detailed amounts prepared were as shown in Table 2, and all numerical values were expressed by “part(s) by mass”.

TABLE 2

| | | Exam- | Exam- | Exam- | Exam- | Exam- | Comaprative | Comparavtive | Comparative |
|---|---|---|---|---|---|---|---|---|---|
| Component | Raw material | ple 1 | ple 2 | ple 3 | ple 4 | ple 5 | Example 1 | Example 2 | Example 3 |
| Component (A) | KY-11 | 76.5 | 76.5 | 76.5 | 76.5 | — | 76.5 | 76.5 | 76.5 |
| | RUA-075 | — | — | — | — | 76.5 | — | — | — |
| | 4-HBA | 15.3 | 15.3 | 15.3 | 15.3 | 15.3 | 15.3 | 15.3 | 15.3 |
| | DCP-A | 8.2 | 8.2 | 8.2 | 8.2 | 8.2 | 8.2 | 8.2 | 8.2 |
| Component (B) | TMMP-20P | 1.2 | 2.4 | 2.4 | 2.4 | 2.4 | 2.4 | 2.4 | 2.4 |
| Component (C) | 184 | 4.1 | 4.1 | 4.1 | 4.1 | 4.1 | 4.1 | 4.1 | 4.1 |
| | TPO H | 3.5 | 3.5 | 3.5 | 3.5 | 3.5 | 3.5 | 3.5 | 3.5 |
| Component (D-1) | TS-720 | 2.1 | 2.1 | 3.2 | 4.2 | 2.1 | 6.3 | — | 3.2 |
| Component (D-2) | RX200 | 4.2 | 4.2 | 3.2 | 2.1 | 4.2 | — | 6.3 | — |
| Component (D′-2) | R972 | — | — | — | — | — | — | — | 3.2 |

| | | | | | | | | |
|---|---|---|---|---|---|---|---|---|
| Total | 115.1 | 116.3 | 116.3 | 116.3 | 116.3 | 116.3 | 116.3 | 116.4 |
| Proportion (%) of component (D) in entire | 5.4 | 5.4 | 5.4 | 5.4 | 5.4 | 5.4 | 5.4 | 5.5 |
| composition | | | | | | | | |
| Total component (D) | 6.3 | 6.3 | 6.3 | 6.3 | 6.3 | 6.3 | 6.3 | 6.4 |
| Component (D-1)/Total comonent (D) (%) | 33 | 33 | 50 | 66 | 33 | 100 | 0 | 50 |
| Component (D-2) (or component (D′-2)/Total | 67 | 67 | 50 | 34 | 67 | 0 | 100 | 50 |
| component (D) (%) | | | | | | | | |

The photocurable compositions of Examples 1 to 5 and Comparative Examples 1 to 3 were each subjected to measurement of the viscosity and the structural viscosity ratio, confirmation of the appearance, and measurement of the turbidity. The results are summarized in Table 3.

### [Measurement of Viscosity and Structural Viscosity Ratio of Photocurable Composition]

Collected was 0.5 ml of each of the compositions, and ejected in a measurement cup. The viscosity was measured with an EHD-type viscometer (manufactured by Toki Sangyo Co., Ltd.) under the following conditions. The result was defined as “viscosity (Pa-s) (also referred to as “viscosity at a rotation rate of 10 rpm”)”. The “viscosity (Pa-s) at a rotation rate of 1 rpm” was measured under the same measurement conditions as described below except that the rotation rate was 1 rpm. The viscosity at a rotation rate of 1 rpm/the viscosity at a rotation rate of 10 rpm was defined as “structural viscosity ratio”. The results are shown in Table 3 below. The viscosity (viscosity at a rotation rate of 10 rpm) is preferably 100 Pa-s or less, more preferably 80 Pa-s or less from the viewpoint of flowability or the like in consideration of handling during procedures. The structural viscosity ratio is preferably 2.5 to 4.5:

### Measurement Conditions

Cone rotor: 3°×R14
Rotation rate: 10 rpm
Measurement time: 3 minutes
Measurement temperature: 25° C. (temperature control by constant-temperature bath).

### [Confirmation of Appearance]

An alkali-free glass plate of 50 mm length×50 mm width×0.7 mm thickness was coated with the composition at a thickness of 0.25 mm, to produce a test piece, and the composition was cured by irradiation with a UV lamp for nails (rated voltage: 100 to 110 V, consumed power at 50 to 60 Hz: 36 W, wavelength: 350 to 400 nm) for 60 seconds. The resultant was visually confirmed according to the following evaluation criteria, and evaluation of “Appearance” was described in Table 3 below. The evaluation “Good” means that use can be made without any problem:
Evaluation criteria
Good: colorless and transparent
Poor: slightly clouded.

### [Measurement of Turbidity]

A SUS304 spacer having a thickness of 1 mm was placed on each of both corners of an alkali-free glass plate of 50 mm length×50 mm width×0.7 mm thickness, and the glass plate was coated with 1 g of the composition. Next, another glass plate was gently placed so as to cause no incorporation of any air bubbles into the composition, to produce a test piece. A portion leaked out of the composition flowed out, and thus the portion leaked out was wiped off. The test piece produced was disposed toward a UV lamp for nails (rated voltage: AC 100 V, consumed power at 50-60 Hz: 36 W, wavelength: 350 to 400 nm), and irradiated twice for 60 seconds, to cure the composition. The test piece was here produced once (n=1). Subsequently, such a test piece containing the composition cured was subjected to measurement with a haze meter NDH2000 manufactured by Nippon Denshoku Industries Co., Ltd., and the result was defined as “turbidity (%)”. The turbidity was measured three times, and the average value was adopted. The “turbidity” is preferably 5.0 to 16.0% from the viewpoint of appearance.

TABLE 3

| | Example | Example | Example | Example | Example | Comparative | Comparative | Comparative |
|---|---|---|---|---|---|---|---|---|
| Component | 1 | 2 | 3 | 4 | 5 | Example 1 | Example 2 | Example 3 |
| Viscosity | 71.3 | 62.5 | 64.2 | 62.5 | 26.0 | 61.8 | 60.9 | 58.0 |
| Structural | 2.6 | 2.7 | 2.8 | 2.9 | 4.2 | 3.1 | 2.1 | 2.2 |
| viscosity | | | | | | | | |
| ratio | | | | | | | | |
| Appearance | Good | Good | Good | Good | Good | Poor | Good | Good |
| Turbidity | 9.0 | 9.8 | 11.7 | 15.3 | 9.1 | 19.5 | 3.2 | 10.2 |

Only the component (D-1) was used in Comparative Example 1 and only the component (D-2) was used in Comparative Example 2. Comparative Example 1, although exhibited thixotropy, exhibited poor appearance and turbidity, and Comparative Example 2, although had a good appearance, exhibited low thixotropy and an improper turbidity. Comparative Example 3 using the component (D′-2) being surface-treated fumed silica where a residue on the surface had no structure represented by the formula 2, although exhibited a low turbidity and a good appearance, did not exhibit an increased structural viscosity ratio. Examples 1 to 5 exhibited high thixotropy, a low turbidity, and also a “Good” appearance, and thus were found to have transparency.

### INDUSTRIAL APPLICABILITY

The present invention allows for not only a structural viscosity ratio capable of providing appropriate control of coating in procedures in the nail field, but also a low turbidity and transparency. The present invention is particularly suitable for an art gel nail for formation of a cubic decoration.

The present application is based on Japanese Patent Application No. 2021-184529 filed on Nov. 12, 2021, the disclosure of which is incorporated by reference in its entirety.

1. A photocurable composition for a nail or an artificial nail, comprising the following components (A) to (D):
Component (A): a compound having a (meth)acryloyl group
Component (B): a polythiol compound
Component (C): a photoinitiator
Component (D): a filling agent containing a component (D-1) and a component (D-2)
Component (D-1): surface-treated fumed silica where a residue on the surface is represented by the following formula 1: 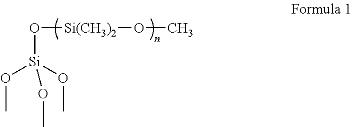
wherein n is an integer of 1 or more;
Component (D-2): surface-treated fumed silica where a residue on the surface is represented by the following formula 2:

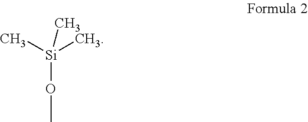

2. The photocurable composition for a nail or an artificial nail according to claim 1, wherein a mass ratio of the component (D-1) and the component (D-2) (component (D-1) component (D-2)) in the entire component (D) is 20:80 to 80:20.

3. The photocurable composition for a nail or an artificial nail according to claim 1, comprising 1.0 to 20.0% by mass of the component (D) relative to the entire composition.

4. The photocurable composition for a nail or an artificial nail according to claim 1, comprising 0.1 to 50 parts by mass of the component (B) and 0.1 to 10 parts by mass of the component (C) based on 100 parts by mass of the component (A).

5. The photocurable composition for a nail or an artificial nail according to claim 1, wherein the component (A) contains a (meth)acrylate oligomer and a (meth)acrylate monomer.

6. The photocurable composition for a nail or an artificial nail according to claim 5, wherein the (meth)acrylate monomer consists only of a monofunctional (meth)acrylate monomer and/or a difunctional (meth)acrylate monomer.

7. The photocurable composition for a nail or an artificial nail according to claim 6, wherein the monofunctional (meth)acrylate monomer is a monofunctional (meth)acrylate monomer having a hydroxyl group.

8. The photocurable composition for a nail or an artificial nail according to claim 6, wherein the difunctional (meth)acrylate monomer is dimethylol tricyclodecane di(meth) acrylate.

9. The photocurable composition for a nail or an artificial nail according to claim 1, for use in an art gel nail.
